(19) United States
(12) Patent Application Publication
(10) Pub. No.: US 2008/0004686 A1
(43) Pub. Date: Jan. 3, 2008

(54) Implantable device with light-transmitting material

(75) Inventors: James B. Hunt, Bloomington, IN (US); Charles W. Agnew, West Lafayette, IN (US); Ram H. Paul JR., Bloomington, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/821,610

(22) Filed: Jun. 25, 2007

Related U.S. Application Data
(60) Provisional application No. 60/818,022, filed on Jun. 30, 2006.

(51) Int. Cl. A61F 2/02

(52) U.S. Cl. 623/1.11; 606/14; 606/8; 607/92; 623/2.11; 623/23.68

(57) ABSTRACT

Endolumenal medical devices for implantation within a body vessel are provided. The medical devices may comprise a light-transmitting area having an adhesive-activating effective transmittance at a wavelength suitable for activating a light-activated adhesive. The light-activated adhesive is preferably positioned on the medical device and may be activated by illumination inside a body vessel with light of a suitable wavelength, including ultraviolet, visible or infrared radiation, to secure the medical device within a body vessel. The light may be provided by a light-emitting catheter within the body vessel, and the medical device may be configured as a radially-expandable valve comprising one or more valve leaflets.

### RELATED APPLICATIONS

This application claims priority to U.S. provisional application Ser. No. 60/818,022, filed Jun. 30, 2006, which is incorporated herein by reference in its entirety.

### TECHNICAL FIELD

The present invention relates to medical devices for implantation in a body vessel. More particularly, the present invention relates to endolumenally implantable medical devices comprising a transmitting area.

### BACKGROUND

Various implantable medical devices are advantageously inserted within various portions of the body. Minimally invasive techniques and instruments for placement of intraluminal medical devices have been developed to treat and repair undesirable conditions within body vessels, including treatment of conditions that affect blood flow such as venous valve insufficiency. In order to minimize the recovery period and reduce the risk of infection and/or rejection, procedures have been developed for delivery and implantation of endoprostheses using minimally invasive procedures. Commonly, such procedures include intraluminal delivery involving percutaneous insertion of an endomedical device by way of a delivery catheter. These less invasive procedures permit delivery and implementation of an endomedical device without the need for replacement of a portion of the vessel, and thus eliminate major surgical intervention and the risks associated therewith.

Various percutaneous methods of implanting medical devices within the body using intraluminal transcatheter delivery systems can be used to treat a variety of conditions. One or more intraluminal medical devices can be introduced to a point of treatment within a body vessel using a delivery catheter device passed through the vasculature communicating between a remote introductory location and the implantation site, and released from the delivery catheter device at the point of treatment within the body vessel. Intraluminal medical devices can be deployed in a body vessel at a point of treatment and the delivery device subsequently withdrawn from the vessel, while the medical device retained within the vessel to provide sustained improvement in valve function or to increase vessel patency. For example, an implanted medical device can improve the function of native valves by blocking or reducing retrograde fluid flow. Alternatively, a prosthetic valve medical device can be implanted to replace the function of damaged or absent native valves within the body.

In order to secure a medical device in place after delivery, the medical device may comprise a securement means, for example sutures, staples or barbs. Additionally, a medical device can optionally include a radially expandable and/or contractible stent or support members positioned to provide an outward radial force securing a medical device against a body vessel. In common usage, after a medical device has been properly positioned within a body vessel, the medical device is expanded to anchor the medical device within the body passageway. Natural cell growth through the wall of the medical device can then further anchor the medical device in place within the body lumen. A medical device for implantation within a body lumen can also be secured with a biocompatible adhesive disposed between the medical device and the wall of a body vessel. The adhesive is preferably selected to provide a desired level of adhesion between the body vessel wall and the implanted medical device in an endoluminal environment so as to bond the exterior surface of the medical device to a surface of the body vessel. The adhesive may be adhered to the medical device prior to delivery of the medical device or may be introduced at the site of implantation within the body vessel. A variety of biocompatible adhesives may be used to secure a medical device within a body vessel in this manner. For example, a medical device may be secured by a light-activated biocompatible adhesive attached to one or more surfaces of the medical device. Activation of the light-activated adhesive within a body vessel can desirably secure a medical device within a body vessel during a delivery procedure.

Desirably, a medical device may be secured within a body vessel by illuminating a light-activated adhesive on the medical device with a suitable light source after deployment of the medical device within a body vessel. For many percutaneous transluminal implantation procedures, light or energy can be delivered from the catheter within a body vessel. For example, light emitting catheters have been designed for providing photodynamic therapy. Light-emitting catheters can include a flexible tube housing a light source, such as an optical fiber coupled to a light diffusion tip, and a transparent portion permitting light from the diffusion tip to exit the catheter and illuminate a portion of a body vessel. In photodynamic therapy, light emitting catheters have been used to selectively kill target cells within a body vessel. However, many prosthetic devices can be formed from materials that are opaque to light at a wavelength suitable for activation of a light-activated adhesive.

What is needed are implantable medical devices configured to permit securing the medical device within a body vessel by exposure to light from a light-emitting catheter. The implantable medical devices provided herein are configured to provide implantable medical devices suitable for percutaneously delivery, such as venous valves or heart valves that may be secured within a body vessel using a minimally invasive catheter-based light-emitting system.

### SUMMARY

This disclosure provides an implantable medical device comprising a material that is transparent to energy at a wavelength and intensity suitable for activating an adhesive positioned to secure the medical device within a body vessel. The adhesive is preferably adhered to a portion of the medical device, or may be introduced within a body vessel at a site of implantation. The activating energy can have any suitable wavelength or intensity, but is preferably visible, infrared or ultraviolet light provided by a catheter having a light source positioned within a body vessel. The medical device is preferably an endolumenal valve placed in the body vessel using a delivery catheter. The delivery catheter may be an expandable balloon catheter and the light source can be provided on the delivery catheter or from a different source. Optionally, the valve can include a radially expandable frame, such as a balloon expandable or self-expanding frame. The valve can assume a compressed configuration around a delivery catheter, and be expanded within the body vessel at a point of treatment.

The light-activated adhesive may be activated from a light source adapted to emit light at a wavelength suitable to activate the adhesive within the body vessel after implantation of the medical device. The medical device may be radially expanded by inflation of an inflatable balloon on a delivery catheter or by permitting the medical device to self-expand within the body vessel. The transparent portion of the medical device and light source can be positioned to illuminate the light-activated adhesive on the expanded medical device within the body vessel. Preferably, the light source is a portion of a catheter having a transparent portion and an inflatable balloon. Alternatively, the light source may be a catheter other than the delivery catheter, or a light source positioned outside the body vessel. For example, a delivery catheter balloon can include a transparent portion oriented to permit energy from a light source within the catheter to pass through both the transparent portion of the catheter balloon and the transparent portion of the medical device, and activate a light-activated adhesive adhered to a portion of the medical device in contact with the wall of the body vessel. The transparent portions of the balloon and the medical device are preferably configured to permit light from the light source within the catheter to activate an adhesive on the valve.

The light-activated adhesive may be applied to a portion of the medical device before, during or after the deployment of the medical device within the body vessel. In one aspect, the light-activated adhesive may be applied to a portion of a medical device comprising extracellular matrix material (such as small intestine submucosa), or other explanted tissue, forming a light-transmitting area. The extracellular matrix material or other explanted tissue may be wet or dry prior to contacting the light-transmitting adhesive, depending on how to best adhere the adhesive to the material.

The light-activated adhesive may be applied to a portion of the medical device, such as an exterior surface, positioned to contact the body vessel wall upon implantation. This may occur prior to introducing the medical device within the body vessel. Optionally, the light-activated adhesive may also be applied to the medical device after deployment of the medical device within the body vessel by releasing the adhesive from a catheter within the body vessel in a manner permitting the light-activated adhesive to adhere to the body vessel wall between the body vessel wall and a portion of the expanded medical device. For example, a liquid light-activated adhesive can be released from a delivery catheter prior to or during the radial expansion of a medical device. The expanded medical device can then be positioned so that light from a light source can pass through the transparent portion of the medical device and activate the light-activated adhesive, thereby adhering the medical device to the wall of the body vessel. Alternatively, the catheter may be used to remove a medical device from a body vessel by providing a light-activated adhesive on the exterior of an expandable balloon positioned along the catheter. The catheter may be positioned within the lumen of a previously implanted medical device, the balloon inflated to place the light-activated adhesive in intimate contact with a portion of the interior surface of the implanted medical device, and activation of the adhesive may adhere the medical device to the balloon. Optionally, the catheter may emit light at a second wavelength selected to weaken attachment of the medical device to the body vessel. Deflation of the balloon and lateral translation of the catheter with respect to the body vessel may radially collapse and remove the medical device from the body vessel.

The medical device is preferably an endolumenal valve or stent graft adapted to be secured within a body vessel using an adhesive that is activated (e.g., solidified) after the valve is placed within a body vessel. The valve can have a portion that is transparent to energy of a wavelength effective to activate the adhesive adhering the valve to the body vessel wall. Preferably, portions of the valve that contact the body vessel upon implantation may be coated with an adhesive that can be activated by an energy wavelength that can pass through the transparent portion. The adhesive can be applied prior to insertion of the valve in a delivery catheter, or can be applied within the body using a portion of a delivery catheter, such as a moveable sheath around the valve. The adhesive is preferably a UV light-activated, visible light-activated, IR-light activated, ultrasonically activated or heat-activated biocompatible material. Alternatively, the adhesive can be formed within the body vessel by combining polymer components or exposing an adhesive material to blood, such as hydrogel polymers that swell to secure the valve within the body vessel.

In one exemplary embodiment, the implantable medical device can be configured as an endolumenal valve having at least one flexible valve leaflet comprising a transparent portion. The valve is preferably radially expandable within a body vessel, and can be delivered from a catheter placed within the body vessel. Preferably, the catheter can include a light source, a transparent portion and an inflatable balloon. The valve may be radially expanded by inflation of the inflatable balloon. The transparent portion and light source can be positioned to illuminate the light-activated adhesive on the expanded valve within the body vessel. For example, the balloon can include a transparent portion oriented to permit ultraviolet energy from a light source within the catheter to pass through both the transparent portion of the catheter balloon and the transparent portion of the valve, and activate a light-activated adhesive adhered to a portion of the valve in contact with the wall of the body vessel. The transparent portions of the balloon and the valve are preferably configured to permit light from the light source within the catheter to activate an adhesive on the valve.

### BRIEF DESCRIPTION OF THE DRAWINGS

FIG. 1A is longitudinal cross section of a light-emitting catheter positioned within a frameless valve; FIG. 1B is an end view of the frameless valve shown in FIG. 1A.

FIG. 2 is a side cut-away view showing a light-emitting catheter positioned within a stent graft.

FIG. 3A is a side view of a light-emitting catheter positioned proximate to a radially expanded valve.

FIG. 3B is a detail view of a portion of the radially expanded valve frame shown in FIG. 3A.

FIG. 4 is a longitudinal cross section of a delivery catheter positioned proximate an implanted prosthetic venous valve within a vein segment.

### DETAILED DESCRIPTION

The following detailed description and appended drawings describe and illustrate various exemplary embodiments of the invention. The description and drawings serve to enable one skilled in the art to make and use the invention.

Definitions

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

The term “light” as used herein refers to radiation of any wavelength suitable for activating a light-activated adhesive material, including radiation with wavelengths in the infrared, visible and ultraviolet regions. As used herein, infrared light includes radiation with a wavelength of between about 1 mm and 750 nm, visible light includes radiation with a wavelength of between about 750 nm and 400 nm, and ultraviolet light includes radiation with a wavelength of between about 400 nm and 10 nm, but preferably between 400 nm and about 200 nm. Accordingly, the term “light” as used herein, unless otherwise indicated, preferably comprising radiation at one or more suitable wavelengths between about 1 mm and 10 nm, preferably between about 1.5 μm to about 200 nm.

The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause an undesirably adverse, long-lived or escalating biological reaction or response, and is distinguished from a mild, transient inflammation which typically accompanies surgery or implantation of foreign objects into a living organism.

A large number of different types of materials are known in the art which may be inserted within the body and later dissipate. The term “bioabsorbable” is used herein to refer to materials selected to dissipate upon implantation within a body, independent of which mechanisms by which dissipation can occur, such as dissolution, degradation, absorption and excretion. The terms “bioabsorbable,” “bioabsorbable,” “bioabsorbable,” or “biodegradable” are used synonymously herein, unless otherwise specified, to refer to the ability of the material or its degradation products to be removed by biological events, such as by fluid transport away from the site of implantation or by cellular activity (e.g., phagocytosis). Only the term “bioabsorbable” will be used in the following description to encompass absorbable, absorbable, bioabsorbable, and biodegradable, without implying the exclusion of the other classes of materials. “Non-bioabsorbable” or “biostable” material refers synonymously to a material, such as a polymer or copolymer, which remains in the body without substantial bioabsorption.

The terms “remodelable” or “bioremodelable” as used herein refer to the ability of a material to allow or induce host tissue growth, proliferation or regeneration following implantation of the tissue in vivo. Remodeling can occur in various microenvironments within a body, including without limitation soft tissue, a sphincter muscle region, body wall, tendon, ligament, bone and cardiovascular tissues. Upon implantation of a remodelable material, cellular infiltration and neovascularization are typically observed over a period of about 5 days to about 6 months or longer, as the remodelable material acts as a matrix for the ingrowth of adjacent tissue with site-specific structural and functional properties. The remodeling phenomenon which occurs in mammals following implantation of extracellular matrix materials, such as submucosal tissue, includes rapid neovascularization and early mononuclear cell accumulation. Mesenchymal and epithelial cell proliferation and differentiation are typically observed by one week after in vivo implantation and extensive deposition of new extracellular matrix occurs almost immediately.

As used herein, the term “body vessel” means any body passage lumen that conducts fluid, including but not limited to blood vessels, esophageal, intestinal, billiary, urethral and ureteral passages.

The term “alloy” refers to a substance composed of two or more metals or of a metal and a nonmetal intimately united, for example by chemical or physical interaction. Alloys can be formed by various methods, including being fused together and dissolving in each other when molten, although molten processing is not a requirement for a material to be within the scope of the term “alloy.” As understood in the art, an alloy will typically have physical or chemical properties that are different from its components.

The medical devices of the embodiments described herein may be oriented in any suitable absolute orientation with respect to a body vessel. The recitation of a “first” direction is provided as an example. Any suitable orientation or direction may correspond to, a “first” direction. The medical devices of the embodiments described herein may be oriented in any suitable absolute orientation with respect to a body vessel. For example, the first direction can be a radial direction in some embodiments.

The terms “frame” and “support frame” are used interchangeably herein to refer to a structure that can be implanted, or adapted for implantation, within the lumen of a body vessel. As used herein, a “support frame” is any structure that is attached to the covering material, for example to hold the covering material in place within a body vessel, including an interior portion of a blood vessel, lymph vessel, ureter, bile duct or portion of the alimentary canal. A “valve support frame,” as used herein, refers to a support frame that forms a portion of a valve means for modifying fluid flow within a body vessel. The valve support frame can have any suitable configuration, but is preferably a radially expandable structure comprising a plurality of struts and bends and enclosing an interior lumen. Preferably, one or more valve leaflets can be attached to the valve support frame.

Medical Device Configurations

This disclosure provides an implantable medical device comprising a material that is sufficiently transparent to energy effective to activate a light-activated adhesive. For example, the transparent material may have a transmissivity to visible, infrared, ultraviolet, ultrasonic or heat energy that is sufficient to activate a light-activated adhesive. The medical device is preferably an endolumenal valve placed in the body vessel using an expandable catheter balloon. Optionally, the valve can include a frame, such as a balloon expandable or self-expanding frame. The valve can assume a compressed configuration around a delivery catheter, and be expanded within the body vessel at a point of treatment.

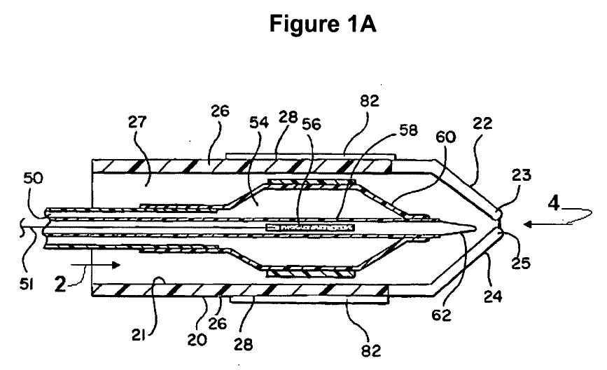

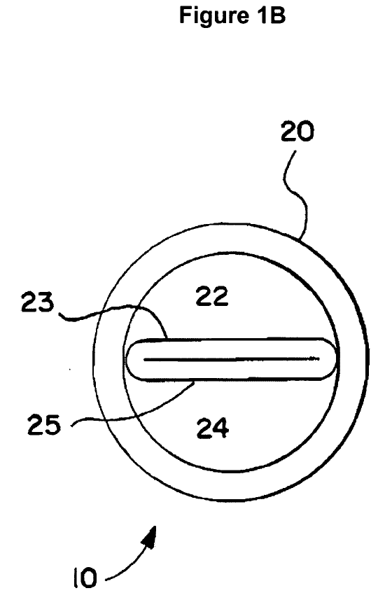

In one exemplary embodiment, the implantable medical device can be configured as an endolumenal valve having at least one flexible valve leaflet comprising a transparent portion. The valve is preferably radially expandable within a body vessel adapted to be delivered from a catheter placed within the body vessel and secured within a body vessel using an adhesive that is activated (e.g., solidified) after the valve is placed within a body vessel. FIG. 1A shows a portion of a medical device delivery system including a light source configured as a radial light-diffusing element 56 inside a catheter 50 positioned within a lumen 27 of a frameless tubular valve 20. The frameless tubular valve 20 may be an endolumenal medical device that comprises a tube of flexible material having a light-transmitting area 28 between a portion of an interior surface 21 defining the lumen 27 and a corresponding portion of the exterior surface 29. A light-activated adhesive 82 may be adhered to the portion of the exterior surface 26 over a light-transmitting area 28. Alternatively, the light-activated adhesive 82 may be applied to other portions of the valve 20 that are not light-transmitting.

The valve 20 is tapered at one end to form a first leaflet 22 and a second leaflet 24 opposably positioned to form a valve orifice between a first edge 23 of the first leaflet 22 and a second edge 25 of the second leaflet 24. FIG. 1B is an end view 10 of the endolumenal valve 20 of FIG. 1A, showing the valve orifice in a closed position, defined by the free edge 23 of the first leaflet 22 and the free edge 25 of the second leaflet 24. The valve 20 can function to regulate fluid flow through a lumen 27 defined by the interior surface 21 of the valve 20. In operation, fluid flow passes through the lumen 27 in an antegrade direction 2 with sufficient force may move the leaflets (22, 24) away from each other, thereby moving the first and second free edge (23, 25) away from each other and opening the valve orifice to permit the fluid flow to proceed. In contrast, fluid flow in the retrograde direction 4 contacts the surface of the leaflets (22, 24), forcing the free edges 23, 25 toward each other, thereby closing the valve orifice and preventing fluid flow into the lumen 27. In this manner, the valve 20 can act as a one-way valve permitting fluid flow in the antegrade direction 2, while substantially blocking fluid flow in the retrograde direction 4. Accordingly, the valve 20 can provide substantially unidirectional flow upon implantation in any body vessel, including within veins, arteries or within the heart.

Portions of the valve that contact the body vessel upon implantation can be coated with an adhesive that can be activated by an energy wavelength that can pass through the transparent portion. The adhesive can be applied prior to insertion of the valve in a delivery catheter, or can be applied within the body using a portion of a delivery catheter, such as a moveable sheath around the valve. The adhesive is preferably a visible light-activated, UV light-activated, ultrasonically-activated or heat-activated biocompatible material. Alternatively, the adhesive can be formed within the body vessel by combining polymer components or exposing an adhesive material to blood, such as hydrogel polymers that swell to secure the valve within the body vessel.

The light-activated adhesive 82 may be activated by any suitable activation means, including illumination with ultraviolet, visible or infrared light energy. “Activation” of the light-activated adhesive refers to any chemical or physical process occurring in the light-activated adhesive occurring during or after interaction with the activation means that permits the light-activated adhesive 82 to function to secure a medical device with respect to the interior wall of a body vessel. Activation processes may include photo-induced sol-to-gel transformation of a polymeric adhesive material driven by photochemical reactions. For example, the light-activated adhesive 82 may be attached to an adhesive surface on a medical device, and may be activated by illumination by light energy causing solidification of the light activated adhesive 82. Solidification of the light activated adhesive 82 may occur by any chemical process, including cross-linking of a polymer induced by ultraviolet light. The activation means may be provided from any suitable source, including light originating within the lumen of a body vessel, from within the lumen 27 of a valve 20, or from outside the body vessel.

The valve 20 is preferably secured within a body vessel by activating a light-activated adhesive 82 present on a surface of the valve. The valve 20 preferably comprises a portion that transmits energy of a wavelength effective to activate the light-activated adhesive 82 used to secure the valve within the body vessel. The light-transmitting area 28 may be configured and positioned with any shape or orientation that permits light passing through the light-transmitting area 28 to be incident upon at least a portion of the adhesive 82. Preferably, the light-transmitting area 28 is positioned between the exterior surface 26 and the interior surface 21 and is radially aligned with a portion of the lumen 27. For example, in a valve 20, light from the light-diffusing element 56 light source within the lumen 27 may pass through the light-transmitting area 28 and impinge upon the light-activated adhesive 82, thereby solidifying the light-activated adhesive 82 adhered to a portion of the exterior surface 26 forming the light transmitting area 28. In the embodiment illustrated by valve 20, the light-activated adhesive 82 is applied directly exterior surface 26. Alternatively, medical device configurations may comprise a light-activated adhesive 82 that is not applied to a portion of a light transmitting area 28. The light-transmitting area 28 may be formed from any material in any configuration that provides an adhesive-activating amount of light to be transmitted to the light-activated adhesive 82.

The thickness, light wavelength, light source, configuration and type of the material forming the light-transmitting area 28 are among the design choices that can be selected to optimize the process of securing the valve 20 within a body vessel by passing light through the light-transmitting area 28 and onto the light-activated adhesive 82. The light-activated adhesive 82 can be applied to any portion of the valve 20 where light from a light source may pass through a light transmitting area 28 and impinge upon the light-activated adhesive 82, and may have any suitable configuration. Preferably, the light-activated adhesive 82 is applied to at least a portion of the exterior surface 26 and/or the interior surface 21 of the valve 20. For example, the light-activated adhesive 82 may be applied as a continuous ring around the exterior surface 26 of a tubular valve 20, or as a plurality of isolated regions on the exterior surface 26.

The valve leaflet preferably includes the light-transmitting area 28 and optionally may also include the light-activated adhesive 82. The valve leaflet can be formed from any suitable material having a desired level of flexibility to permit the leaflet to move in response to fluid flow or changes in pressure within a body vessel. The valve leaflet can comprise one or more materials. Preferably, a valve leaflet comprises a material that is bioremodelable. A bioremodelable material can provide an extracellular matrix that permits, and may even promote, cellular invasion and ingrowth into the material upon implantation. Examples of bioremodelable materials include reconstituted or naturally-derived collagenous materials. Preferably, the material is an extracellular matrix materials (ECMs) possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa. The submucosa or other ECM material used in the present invention may also exhibit an angiogenic character and thus be effective to induce angiogenesis in a host engrafted with the material. The bioremodelable material having in vivo angiogenic properties may be identified using a subcutaneous implant model to determine the angiogenic character of a material, as disclosed in C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268. Submucosa or other ECM materials of the present invention can be derived from any suitable organ or other tissue source, usually sources containing connective tissues. Small intestine submucosa (SIS), such as porcine SIS, is one particularly preferred ECM. Submucosa or other ECM tissue used in the invention is preferably highly purified, for example, as described in U.S. Pat. No. 6,206,931 to Cook et al., incorporated herein by reference in its entirety. Preferably, the ECM material forms at least a portion of the light-transmitting area.

The valve leaflet may also be formed from or include explanted biological tissue, such as aortic tissue, that is treated in a manner that improves the biocompatibility of the tissue for an intended use. For example, the tissue may be treated to improve resistance to post-implantation mineralization. One preferred method is described in U.S. Pat. No. 5,595,571 (Filed Apr. 18, 1994), incorporated by reference herein in its entirety, which involves exposing biological material including cellular and non-cellular structural components to a buffered solution having a pH in the range from about 5.0 to about 8.0 and a temperature in the range from about 12° C. to about 30° C. for a sufficient amount of time to facilitate the degradation of cells by autolytic enzymes within the cells, whereby at least one region of the biological material is rendered substantially acellular while preserving the overall structural integrity and non-cellular structural components of the biological material The exposure occurs prior to any fixation of the biological material. Other suitable tissue treatments are described in the following references, all of which are incorporated herein by reference in their entirety: U.S. Pat. Nos. 5,720,777, 5,843,180 and 5,843,181 (Biological Material Pre-fixation Treatment); U.S. Pat. No. 4,798,611 (Enhancement of Xenogeneic Tissue by treatment with glutaraldehyde and then irradiation); U.S. Pat. No. 4,813,958 (Crosslinked anisotropic mammalian diaphragm in surgical reconstruction); U.S. Pat. No. 3,966,401 (Tissue for Implantation so as to Provide Improved Flexibility by Tissue subjecting tissue to tanning fluid when under pressure until the tissue assumes a natural configuration during tanning in Tanning fluids including 4% formaldehyde and 2% glutaraldehyde); U.S. Pat. No. 4,800,603 (Tissue Fixation with Vapor by subjecting tissue to a vapor of a fixative while the tissue is unstressed); and U.S. Pat. Nos. 4,813,964 and 4,813,958 (Crosslinked anisotropic xenogeneic diaphragm tissue in flexor tendon pulley reconstruction, such as a method of tissue replacement for nonfunctional flexor tendon pulleys including replacing the flexor tendon pulleys with anisotropic, crosslinked mammalian, bovine or porcine diaphragm which is characterized in that the diaphragm has one smooth side and one fibrous side, the smooth side being placed against the flexor tendon). Preferably, the explanted tissue explanted tissue is pre-treated by performing at least one of the following steps: maintaining the explanted tissue at a pH in the range from about 5.0 to about 8.0 and a temperature in the range from about 12° C. to about 30° C. for a sufficient amount of time sufficient to effect the degradation of at least a portion of the cells by autolytic enzymes within the cells; contacting the explanted tissue with a chemical cross-linking agent and then irradiating with X-ray or gamma radiation; contacting the explanted tissue with a tanning fluid including formaldehyde or glutaraldehyde; or placing tissue explanted tissue within an atmosphere of substantially unpressurized vapor of containing glutaraldehyde, and maintaining the tissue within the atmosphere of substantially unpressurized vapor in a manner sufficient to provide substantially uniform application of the fixative solution for a period of time to cause the desired fixation of said tissue.

The valve leaflet can also be formed from a sheet of a first material attached to a second material, such as a two-layer composite. The multilayer composite valve leaflet material can be formed by pressing two layers of material together, sewing or applying an adhesive between the layers. Alternatively, a first material can be formed by a first sheet of material having a resected portion to form a “window” to permit light to pass through the window portion. A second material can be attached to the cover the window. The first and second material can have the same or different transmittance for light at a wavelength suitable for activating the light-activated adhesive.

Preferably, light energy for activating the light-activated adhesive 82 may originate from a catheter 50 positioned within the lumen of a body vessel, as shown in FIG. 1A. The catheter 50 may optionally be adapted to deploy the valve 20, for example by expanding the balloon 60. Alternatively, the catheter 50 may be positioned within a body vessel proximate a previously implanted valve 20. The catheter 50 includes a light source comprising an optical fiber 51 coupled to a radial light-diffusing element 56 positioned within a central lumen defined by an interior tube 58 formed from a material having a desired transmittance to the light from the light source to permit activation of a light-activated adhesive 82. The catheter 50 may also include a balloon 60 positioned proximate the distal tip 62, which comprises an inflation lumen 54. The inflation lumen 54 may have an annular configuration positioned around the central lumen 58.In operation, the valve 20 may be crimped around the balloon 60 and placed at a point of treatment within a body vessel. The balloon 60 may be inflated by expanding the inflation lumen 54 to radially expand the valve 20 until at least a portion of the light-activated adhesive 82 contacts the inner wall of the body vessel. Light energy from the light source, such as the radial light diffusion element 56, may be provided for a duration and intensity sufficient to activate the light-activated adhesive 82 and secure the valve 20 within the body vessel. Next, the balloon 60 may be deflated, and the catheter 50 withdrawn from the body vessel. The catheter 50 is preferably configured and positioned to prevent undesirably adhesion between the light source and the light-activated adhesive 82. Optionally, the catheter 50 may be adapted to release the light-activated adhesive 82 into the lumen of the body vessel.

In a first aspect, the catheter 50 may be used to secure a deployed valve 20 within a body vessel, as described above. However, other uses for the catheter 50 include removal of a valve 20 from a body vessel. For example, in a second aspect, a light-activated adhesive may be positioned on the exterior surface of the balloon 60. Expansion of the inflation lumen 54 may position the light activated adhesive 82 in intimate contact with the interior surface 21 of the valve 20. Activation of the light activated adhesive by the light source may adhere to the interior surface 21 of the valve 20 to the balloon 60. The valve 20 may be collapsed by deflating balloon 60, pulling the valve 20 radially inward away from the wall of the body vessel. Optionally, the light source may emit light at a first wavelength to weaken attachment of a previously implanted valve 20 to a body vessel wall (e.g., to break down an adhesive previously solidified), and emit light at a second wavelength to activate a light-activated adhesive between the exterior surface of the balloon 60 and the interior surface 21 of the valve 20.

The catheter 50 is one example of an endolumenal light source placed within a body vessel. However, light may be emitted from any light source. Preferably, the light source is an endolumenal medical device comprising a catheter operatively joined to a light source, for example by placing an optical fiber and a light diffusion element within a lumen of the catheter. For example, photodynamic therapy light delivery catheters may be used to provide light for activation of the light-activated adhesive. Suitable endolumenal light sources include the light-emitting catheters disclosed in U.S. Pat. No. 6,086,558 (filed Dec. 29, 1998), PCT applications WO 90/00914 and WO 90/00420 (Rowaland et al.), Nseyo et al., Urology 36: 398-402 (1990), U.S. Pat. Nos. 4,998,930, 5,354,293 and 5,125,925, and Panjehpour et al., Lasers and Surgery in Medicine 12: 631-638 (1992). Alternatively, the light source may be located outside the body of a patient or outside the body vessel containing the valve 20.

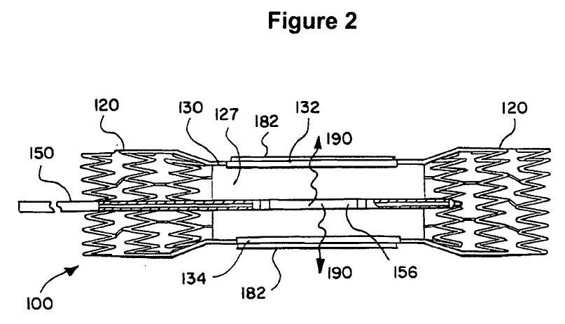

The medical device need not be a valve 20, as shown in FIGS. 1A-1B, but may alternatively be configured as any endolumenal medical device that can be secured with a light-activated adhesive, including stents, stent grafts, vascular grafts, catheters, shunts, wraps and the like. Referring to FIG. 2, a medical device 100 is configured as a radially self-expanding stent graft comprising a tubular graft material 130 enclosing a portion of a radially self-expanding frame 120 formed by a plurality of hoops having a plurality of interconnected struts and bends forming a “zig zag” configuration. The frame 120 may be formed by any biocompatible material, but is preferably a self-expanding nickel-titanium material such as NITINOL.

The graft 130 comprises a light-transmitting area 132 between an interior surface of the graft 130, defining a cylindrical lumen 127. The graft 130 may be formed from any suitable material, such as a polyimide (e.g., nylon) mesh or polytetrafluoroethylene (PTFE). Preferably, the graft 130 material is selected to permit an adhesive-activating transmittance at a wavelength suitable for activating a light-activated adhesive 182. The light-activated adhesive 182 may be applied to a portion of the exterior surface and/or interior surface to form an adhesive surface 134. The adhesive surface 134 is preferably at least a portion of a light-transmitting area 132 between the interior and exterior surfaces of the graft 130. The light-activated adhesive 182, light-transmitting area 132 and adhesive surface 134 may comprise any suitable material, including those discussed above with reference to valve 20 in FIGS. 1A-1B.

The medical device 100 may be positioned within a body vessel by any suitable method, including deployment from a delivery catheter. The medical device is preferably self-expanding. A self-expanding medical device 100 may be retained in a radially compressed configuration within a delivery catheter by a moveable sheath. The moveable sheath may be moved longitudinally relative to the medical device 100 to permit the medical device 100 to radially self-expand at a point of treatment within a body vessel. Once implanted, the medical device 100 may be secured by activation of the light-activated adhesive 182. A catheter 150 comprising a light source 156 may be positioned within the lumen 127 of the medical device 100. The light source 156 may be any suitable means for providing light at a desired wavelength that is effective to activate the light-activated adhesive 182. Optionally, the catheter 150 may be provided without a balloon portion. The light-activated adhesive 182 may be activated by light energy 190 passing through the light-transmitting area 132 and into the adhesive surface 134. The light energy 190 may be provided at a wavelength, intensity and duration adequate to sufficiently solidify the light-activated adhesive 182. Subsequently, the catheter 150 may be removed from the body vessel. Preferably, the catheter 150 is also a delivery catheter comprising a deployment portion adapted to retain the medical device 100 during delivery, and a sheath moveable to deploy the medical device 100. Alternatively, the catheter 150 may be placed within the lumen 127 of a previously deployed medical device 100 within a body vessel. Optionally, a portion of the frame 120 may be formed from a light-conducting material, such as an optical fiber segment, to form a transmitting surface.

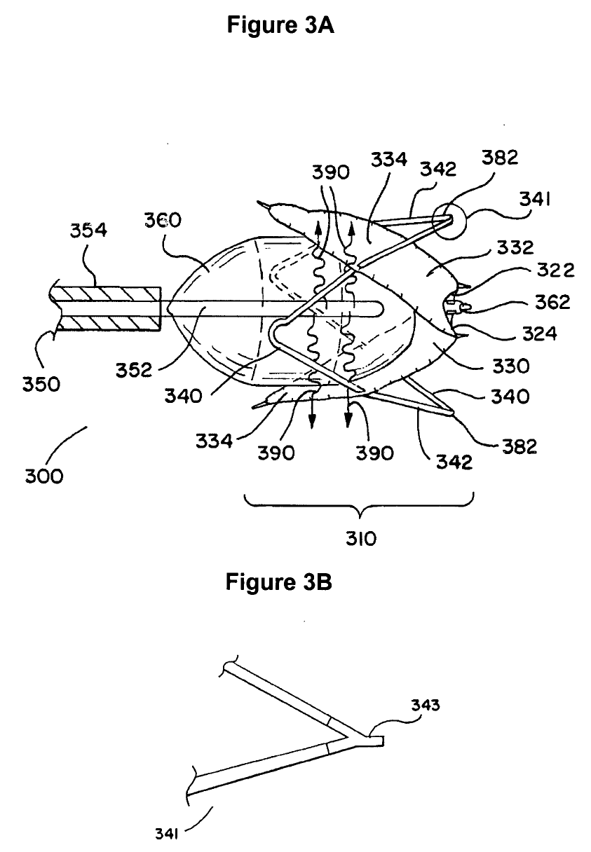

Other medical device embodiments provide adhesive surfaces and transmitting surfaces positioned at separate portions of the medical device. FIG. 3A shows a delivery system 300 comprising a valve 310 circumferentially positioned around a delivery catheter 350. The valve 310 comprises a first valve leaflet 330 and a second valve leaflet 332 attached to a radially expandable frame 340. A light-activated adhesive is adhered to portions of the expandable frame 340 to form a plurality of adhesive surfaces 342 on the frame 340. The first leaflet 330 is attached to the frame 340 on two sides, and includes a third side forming flexible free edge 324; the second leaflet 332 is similarly dimensioned and attached to the frame 340 on two sides and includes a third side forming flexible free edge 322. Together, the free edge 324 and free edge 322 define the valve orifice 362, which is moveable to regulate fluid flow across the valve 310 within a body vessel, in a manner substantially similar to the function of valve 20 described with respect to FIGS. 1A-1B. The first leaflet 330 and the second leaflet 332 each form light-transmitting surfaces 334 that permit an adhesive-activating amount of light to pass through each leaflet 330, 332. Alternatively, a portion of the first leaflet 330 or the second leaflet 332 can be form a transmitting surface “window” within the leaflet 330, 332. The transmitting surface may be made from a transmitting material, or may be a hole in the leaflet 330, 332 permitting light to pass through the hole. For example, a transmitting material may be sewn over a hole cut in the leaflet 330, 332 to provide a transmitting surface when the leaflet 330, 332 is not formed from a transmitting material. The valve 310 may be positioned within a body vessel by radially expanding the valve 310. The frame 340 is moveable from a radially compressed configuration to the radially expanded configuration shown in FIG. 3A, for example by expansion of a balloon 360 on the catheter 350, or by self-expansion. Optionally, the catheter 350 may include a retractable sheath 354, which may be moved longitudinally relative to the compressed valve 310 to permit expansion of the valve 310. In the radially expanded configuration, the light-activated adhesive is placed in intimate contact with the wall of a body vessel and the adhesive surfaces 340 are radially aligned with at least a portion of the transmitting surfaces 334. The catheter 350 preferably includes a light source 352 adapted to emit light 390 at one or more wavelengths selected to activate the light-activated adhesive. The emitted light 390 may pass through the transmitting surfaces 334 of the first valve leaflet 332 and the second valve leaflet 330 to impinge on the adhesive surface 342 of the frame 340. After deploying the valve 310 and activating the light-activated adhesive, the catheter 350 may be removed from the body vessel, leaving the valve 310 secured within the body vessel. FIG. 3B shows a detail view of a bend portion 341 of the frame 340 shown in FIG. 3A. A light diffusing mesh 343 comprising a light-activated adhesive is attached to the frame 340. The light diffusing mesh 343 may be formed from any suitable material, such as copolymer or blend of polyethylene and polypropylene, and can be bonded to the frame 340 during manufacture of the medical device. A light activated adhesive can be impregnated in the light diffusing mesh 343 or can be released from a delivery catheter at a point of treatment within a body vessel in a manner permitting contact of the light-activated adhesive and the light diffusing mesh 343 within the body vessel.

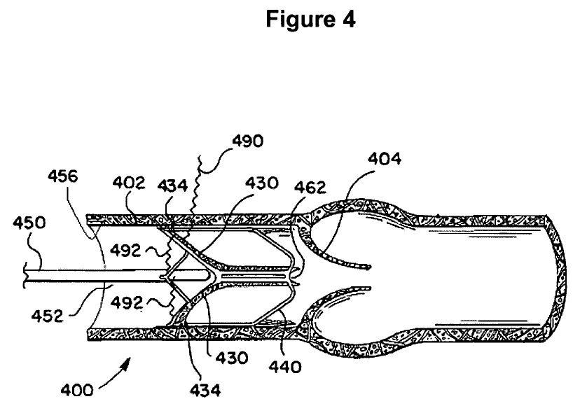

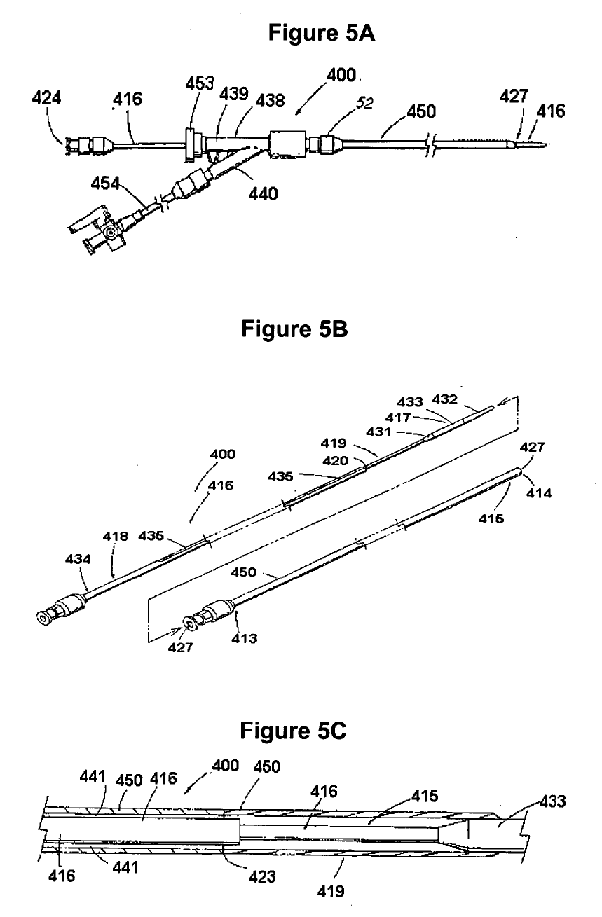

Still other medical device embodiments, the light for activating the light-activated adhesive may be provided from outside the body vessel. Referring to FIG. 4, an implanted prosthetic venous valve 400 is shown within a body vessel 402, positioned proximate a defective native venous valve 404. The prosthetic valve 400 is positioned proximate to a native venous valve 404, preferably upstream from the native venous valve 404 within a vein. The prosthetic venous valve 400 comprises a pair of valve leaflets 430 separately attached to a radially self-expanding frame 440. Each valve leaflet 430 extends from a base 434 to a free edge; the pair of free edges define a valve orifice 462. The valve 400 functions to substantially permit unidirectional fluid flow by movement of the valve leaflets 330 in a manner similar to the valve 20 described with respect to FIGS. 1A-1B. The external side of the base 434 of each leaflet 430 is coated with a light-activated adhesive in contact with the interior surface 456 of the body vessel 402. An endolumenal light source 450 (such as a PDT balloon catheter) may be positioned within the lumen 452 of the body vessel 402. Accordingly, the portion of the base 434 to which the light-activated adhesive is applied forms a transmitting surface radially aligned with an adhesive surface, as light may pass through the base 434 to activate the light-activated adhesive. The light-activated adhesive may be activated by a first light 490 from a light source positioned external to the body vessel 402. The first light 490 may be selected to pass through the wall of the body vessel 402 and to minimize tissue damage, such as an infrared light. Optionally, a second light 492 may be provided from inside the body vessel 402, for example from a catheter 450 comprising a light emitting source 452. In one aspect, the first light 490 may be at a different wavelength from the second light 492. The first light 490 and the second light 492 may be selected to interact with each other to intensify the energy delivered to the adhesive surface.

The invention includes other embodiments within the scope of the claims, and variations of all embodiments, and is limited only by the claims made by the Applicants. Additional understanding of the invention can be obtained by referencing the detailed description of embodiments of the invention, below, and the appended drawings.

Light-Activated Adhesive Materials

An endolumenal medical device comprising a transmitting surface is preferably secured with a suitable light-activated adhesive. The light-activated adhesive may be disposed on the exterior surface of a medical device to form an adhesive surface before, or during implantation of the medical device in a body vessel. The light-activated adhesive is preferably selected to be activated within an intraluminal environment so as to bond the adhesive surface to the intraluminal surface of the body vessel. The light-activated adhesive is preferably biostable, but may also be bioabsorbable, and may be selected from any curable polymer adhesive, including photodynamically cured adhesives that are activated (e.g., solidified) by exposure to ultraviolet, infrared or visible radiation. Suitable light-activated adhesives include cyanoacrylates, polyurethanes, silicones, (meth)acrylates and combinations thereof. The light-activated adhesive may optionally function as a biological sealant capable of bonding an adhesive surface to a body vessel surface, such as fibrin, collagen, poly(L-glutamic acid), gelatin based hydrogels, N-vinyl pyrollidone and mixtures thereof. A light-activated adhesive material may be activated by irradiation with light at any suitable wavelength.

The light-activated adhesive material may be activated by irradiation with ultraviolet light. Desirably, the UV-curable adhesive can form a solid upon irradiation with UV radiation within a body vessel. The UV-curable adhesive is preferably flexible, elastomeric, fast curing, highly adhesive to tissue in vivo and biocompatible. The UV-curable adhesive is preferably rapidly cured, for example within about 3 minutes, and more preferably within less than 1 minute of UV irradiation at a suitable wavelength. The adhesive strength of the activated adhesive is preferably larger than internally generated stress within the body vessel, such as blood pulsatile forces. For example, the light-activated adhesive may be an ultraviolet (“UV”)-curable adhesive, including polyurethanes, epoxies, acrylics, polyethers and mixtures thereof. Multifunctional polyether polymer adhesives, for example, can be activated by photo-induced radical polymerization within a body vessel. The light-activated adhesive may undergo activation by a photo-induced sol or liquid-to-gel transformation. One suitable difunctional biostable polyether-based adhesive comprises diacrylated polyether polymers such as poly(ethylene glycol) (PEG), poly(propylene glycol) (PPG), or the random copolymer PEG-PPG, and may be activated by conversion to water swellable gels by UV or visible irradiation in the presence of a suitable initiator (such as benzophenone, xanthene dyes, or benzoin). Examples of these difunctional polyether-based adhesives are provided in Itoh et al., “Development of photocurable medical-use resins: molecular design and properties,” Jpn. J. Artif. Organs, 18, 132-136 (1989), incorporated herein by reference. Alternatively, the adhesive may comprise a diacrylated macromere comprising a PEG center block and oligomers of alpha-hydroxy acids (such as poly(lactic acid) or poly(glycolic acid)) extending from either end of the PEG center block and capped with acrylate groups, for example as disclosed in Hubbell et al., “Bioerodible hydrogels based on photopolymerized poly(ethylene glycol)-co-poly(alpha hydroxyl acid)diacrylate macromers,” Macromol. 26, 581-587 (1993), incorporated herein by reference. Other suitable adhesives may be activated by cross-linking via intermolecular photodimerization of cinnamate, coumarin or thymine, which may be derivatized in thermally denatured collagen (gelatin), such as the adhesives described by Nakayama et al., “Novel surface fixation technology of hydrogel based on photochemical method: heparin-immobilized hydrogelated surface,” J. Polym. Sci. Polym. Chem. Ed., 31, 977-982 (1993).

In a first aspect, the light-activated adhesive is a UV-activated adhesive comprising a photoreactive gelatin and a multifunctional macromolecule, such as poly(ethylene glycol)diacrylate (PEGDA). The photoreactive gelatin may be formed by reaction with a photoreactive molecule to form a photoreactive group attached to the gelatin. The photoreactive gelatin may be prepared by coupling the amino groups of lysine residues of gelatin with a carboxyl substituent on a benzophenone derivative or a carboxyl residue of xanthene dye in the presence of a water soluble carbiimide (WSC) as a condensation agent. Examples of suitable photoreactive groups include UV-reactive benzophenone or a visible light-reactive xanthene dye, such as fluoroscein sodium salt, erosin Y or rose bengal. The photoreactive molecule and the gelatin may be reacted in a solvent such as phosphate buffered saline (PBS) to form the photoreactive gelatin, followed by isolation of the photoreactive gelatin by dialysis and lyopholization to form a powder. The number of photoreactive groups added to the gelatin can be varied by changing the amount of the photoreactive molecule reacted with the gelatin. Typically, about 4-32 groups of benzophenone are added per mol of gelatin, or about 11 groups of xanthene dye per mol of gelatin. Preferably, UV-activated photoreactive gelatins comprise about 30-35 groups of benzophenone per mol of gelatin. A light-activated adhesive may be formed by combining a suitable amount of the photoreactive gelatin adhesive with PEGDA. The adhesive may optionally further comprise ascorbic acid as a reducing agent. Adhesives comprising the photoreactive gelatin and PEGDA are typically activated by irradiation with UV or visible light for about 1 minute. Preferably, the adhesive composition comprises about 5% to about 30%, more preferably about 10% to about 20%, of the photoreactive gelatin, and optionally further comprises up to about 20% PEGDA. Most preferably, the adhesive comprises about 10% to about 20% photoreactive gelatin and about 10% PEGDA. The PEGDA preferably has a molecular weight of about 1000 to about 10000, more preferably between about 1000 and 4000, and most preferably about 1000, 2100 or 3900. Increasing the amount of photoreactive gelatin and/or PEGDA typically increases the viscosity of the adhesive. A small amount of ascorbic acid, such as about 0.3%, can be added to the adhesive as a reducing agent. UV-irradiation of saline solution comprising the photoreactive gelatin and PEGDA for about 15 to about 60 seconds may produce a suitable gel adhesive. Surgical tissue adhesives comprising photoreactive gelatins may be suitable as light-activated adhesives, including the light-activated photoreactive gelatin adhesives described in Nakayama et al., “Photocurable Surgical Tissue Adhesive Glues Composed of Photoreactive Gelatin and Poly(ethylene glycol)Diacrylate,” J. Biomed. Mater. Res. (Appl. Biomater) 48, 511-521(1999), the complete disclosure of which is incorporated herein by reference.

In a second aspect, the light-activated adhesive is a UV-activated adhesive comprising N-vinyl pyrrolidone (NVP). UV-activated adhesive may be prepared by reacting NVP with various difunctional comonomers suitable to react with amine, hydroxyl, or carboxylic acid groups of proteins in the wall of a body vessel, thereby securing a medical device within the body vessel. Preferred comonomers comprise anionic, imide, epoxy or isocyanate reactive groups that provide a free radical polymerizable vinyl group and a tissue reactive group. Examples of suitable comonomers include 2-acrylamido-2-methyl-1-propane sulfonic acid (AMPS), vinylsuccinimide (VS), 2-isocyanatoethyl methacrylate (IEM) or glycidyl acrylate (GA). Typically, the adhesive composition may be formed by mixing suitable amounts of the NVP with a comonomer in a solvent such as water or 1-methyl-2-pyrrolidinone in the presence of a small amount (e.g., 2 wt %) of a photoinitiator such as hydroxycyclohexyl phenyl ketone. Optionally, the adhesive composition may further comprise a crosslinking agent such as polyethylene glycol 600 dimethacrylate (PEG600DMA) or ethylene glycol dimethacrylate (EGDMA). The amount of NVP and comonomer can be varied, but adhesives typically contain about 10 to about 40 wt %, preferably about 25-35 wt %, of each component. One particularly preferred adhesive comprises about 40 wt % NVP and about 30 wt % IEM and up to 2 wt % PEG600DMA or EGDMA crosslinking agent combined in NMP solvent with a suitable photosensitizer (e.g., about 2 wt % N,N -Diethyl-2,3-dihydroxy-terephthalamide). Another particularly preferred adhesive comprises about 40 wt % NVP and about 35 wt % VS and up to 2 wt % PEG600DMA or EGDMA crosslinking agent combined in NMP solvent with a suitable photosensitizer (e.g., about 2 wt % N,N-Diethyl-2,3-dihydroxy-terephthalamide). These and other UV-activated adhesives comprising NVP are described in Kao et al., “UV Curable Bioadhesives: Copolymers of N-Vinyl Pyrrolidone,” J. Biomed. Mater. Res., 38, 3, 191-196 (1998), which is incorporated herein by reference in its entirety.

In a third aspect, the light-activated adhesive is a UV-activated adhesive comprising the reaction product of acryloyl chloride and various diol crosslinking agent to form a UV-curable monomer such as 2,2-bis(4-ethoxyacrylatephenyl)propane (HEPAC), 2,2-bis(4-ethoxy-acrylatephenyl)6-Fpropane (HEPFAAC), 2,2-bis(4-ethoxyacrylate-3,5-dibromophenyl)propane (TBHEPAAC) or 5-tert-butyl-1,2-bis(4-ethoxy-acrylate benzoyl)benzene (tBuHEPBAC). The adhesive monomers can be formed by combining acryloyl chloride and a diol crosslinking agent in reaction mixture with a catalytically effective amount of triethylamine (TMA) (about 3 g/mol) in a suitable solvent, such as tetrahydrofuran (THF). For example, HEPAC may be formed by reacting about 3 g/mol acryloyl chloride, 5/mol 2,2-bis(4-β-hydroxyethoxyphenyl)propane (HEPA), 3.2 g/mol TEA and 10 mL THF; HEPFAAC may be formed by reacting 2.25 g/mol acryloyl chloride, 5 g/mol 2,2-bis(4-β-hydroxyethoxyphenyl)hexafluoropropane (HEPFA), 2.38 g/mol TEA and 10 mL THF; TBHEPAAC may be formed by reacting 1.5 g/mol acryloyl chloride, 5 g/mol bishydroxyethyl ether of TBPA (TBHEPA), 1.6 g/mol TEA and 10 mL THF; and tBuHEPBAC may be formed by reacting 2.93 g/molacryloyl chloride, 7.5 g/mol 5-tert-butyl-1,3-bis(4-hydroxyethoxybenzoyl)benzene (tBuHEPB), 3.27 TEA and 10 mL THF. Preferably, the adhesive compositions comprise about 10 wt % of the diol crosslinking agent.

In a fourth aspect, the light-activated adhesive material may be activated by heating, for example with infrared radiation. Preferably, the infrared-activated adhesive is activated by illumination of the adhesive with light of a wavelength at which the adhesive material readily absorbs, without significant absorption of energy by adjacent tissue or body fluids. U.S. Pat. No. 6,176,871, filed Oct. 14, 1998, discloses examples infrared activated adhesives. Still other examples of light-activated adhesives include Cationic Type V157 and V197 epoxies from Electronic Materials, which may be activated by irradiation with long-wave UV light in the range of 325-380 nm or with visible light in the range of 400-500 nm. The Emcast V157 and V197 UV and Visible Light Curable Epoxies from Electronic Materials are cationic epoxies that cure to a flexible consistency, and may be activated by irradiation with long wave UV light in the range of 325-380 nm or with visible light in the range from 400-500 nm.

In a fifth aspect, the light-activated adhesive material may be a material that may become moldable or molten at a temperature that is not significantly injurious to tissue or surrounding physiological fluids if heated within a body vessel to a temperature slightly above body temperature. Preferably, the material becomes moldable at a temperature above about 40° C., including materials described in U.S. Pat. No. 6,176,871, filed Oct. 14, 1998. The moldable material may function as an infrared-light activated adhesive heated by irradiation with infrared radiation at an appropriate wavelength. The infrared-light activated adhesive may further comprise a material that readily absorbs energy from infrared radiation or may comprise a polymeric material with a chromophore that readily absorbs the infrared radiation. Other materials such as fibrin may also be used as the light-activated adhesive. Fibrin may be activated by infrared and/or ultraviolet radiation, as disclosed in U.S. Pat. No. 6,818,008.

A light-activated adhesive may optionally further comprise a chromophore selected to absorb light at a light activating wavelength. The chromophore may be a dye or pigment compounded with the polymer, such as indocyanine green (IG), or ethyl eosin (EE). Examples of suitable chromophores that may be included in the light-activated adhesive, and the associated maximum absorption wavelength, include: Acramine Yellow (420 nm), Acridine Orange (489 nm), Fluorescein (491 nm), Eosin Y (514 nm), Methylene Blue (661 nm), Jenner stain (651 nm), Prussian Blue (694 nm), Indocyanine Green (775 nm), Erythrosin B (525 nm), Eosin Y (514 nm), Acridine (358 nm), and Prussian blue (260 nm). Preferably, the light is provided by a light-emitting catheter positioned within a body vessel, although light may also be provided from outside the body vessel.

The light-activated adhesive may be applied to any surface of the medical device by any suitable method, including spray coating, dip coating, transfer from a tape and the like. The adhesive may be provided longitudinally along the length and/or circumferentially about the medical device to form one or more adhesive surfaces, which may be positioned on a medical device frame or on a material attached to the frame, such as a valve leaflet light-transmitting material.

Light-Transmitting Materials

Preferably, a medical device can comprise a light-transmitting material forming a transmitting surface. The transmitting surface can be any portion of a medical device, including at least a portion of a medical device frame and/or a laminar material such as a valve leaflet or tubular frameless valve. The light-transmitting material may be any material providing a desired transmittance at a wavelength suitable for activating a light-activated adhesive. A particularly preferred light-transmitting material is a remodelable material described above, including an ECM such as small intestine submucosa (SIS), characterized with a desired level of light-transmittance. Other examples of light-transmitting materials include a polymer is selected from the group consisting of: aliphatic polyacrylates, silesesquioxanes, alkyl-substituted silicones and vinyl ethers, polyethylene, polycarbonate, acrylic, polyurethane, Teflon, or PVC. In one aspect, a portion of a frame functions as a transmitting surface. For example, a frame may comprise a non-expanding portion that is transparent to light or that conducts light, such as a hollow optical fiber segment. Alternatively, a light-transmitting frame portion may be incorporated into a light transmitting material such as poly(methylmethacrylate) (PMMA), or a fluorinated derivative thereof. For instance, a strand of light-emitting fiber may be adhered to or woven into a valve leaflet to form a portion of a light-transmitting material.

Preferably, the transmitting surface may be a graft material, valve leaflet or any portion thereof comprising a UV-transmissive material, such as a thin flexible film of UV-transmissive polyethylene polymer. For example, a thin film of polyethylene with a thickness of 0.002 inches (0.05 mm) transmits up to 80% of the ultraviolet light emitted by a light source having a wavelength in the range of 220 to 310 nm. Even polyethylene films up to 0.01 inches (0.25 mm) thick can transmit over 50% of the ultraviolet light. UV-light transmitting polymers are also described in U.S. Pat. No. 5,620,495, filed Aug. 16, 1995, and U.S. Pat. No. 6,222,973, filed Jan. 15, 1999. In another aspect, a medical device can comprise a first light-transmitting material forms a valve leaflet wrapped around a second light-transmitting material that forms a portion of a support frame. In another aspect, a medical device may include a first light-transmitting material forming a valve leaflet wrapped around a second light-transmitting material that forms a portion of a support frame.

In yet another aspect, the transmitting surface may be an extracellular matrix material (ECM), such as small intestine submucosa (SIS), as described above. Preferably, the ECM has a thickness permitting at least about 5% transmittance of light at a wavelength suitable for activating an adhesive. More preferably, the ECM has a thickness permitting at least about 8%, 10%, or greater, transmittance of ultraviolet light. For example, the transmitting surface may be a transmitting area of SIS having a thickness of about 0.0010 to 0.0050-inch, about 0.0015 to 0.0030-inch, or about 0.0015 to 0.0020-inch thick, including any thicknesses therebetween. In one aspect, the light-transmitting area is formed from explanted biological tissue, such as aortic tissue, that is treated in a manner that improves the biocompatibility of the tissue for an intended use (e.g., as described above with respect to valve leaflets). The explanted tissue may function as a graft or a valve leaflet material in addition to forming a light-transmitting area. Optionally, transmitting surface may also be formed as a composite of particles of light-transmitting material impregnated in a polymer carrier and formed in a suitable shape and thickness to provide a transmitting surface. The light-transmitting particles may be UV-transmissive particles may improve the light transmissivity, and may include UV-transmissive materials such as quartz, fused silica, UV-transmissive glass, or ceramic.

Implantable Frames

Any suitable support frame can be used as the support frame in the medical device. The support frame may be a radially self-expanding frame configured to provide a stenting function by exerting a radially outward force on the interior of the body vessel in which the medical device is implanted. Any suitable stent configuration can be used as the support frame. The frame configuration chosen will depend on several factors, including the vessel in which the medical device is being implanted, the axial length of the treatment site, the number of medical devices desired, the inner diameter of the body vessel, and the delivery method for placing the support frame. The support frame is preferably a radially expandable support frame having a radially compressed and a radially expanded configuration, allowing the medical device to be delivered to and implanted at a point of treatment using percutaneous techniques and devices. The support frame can be either balloon- or self-expandable.

The support frame can be formed from a variety of materials, and need only be biocompatible or able to be rendered biocompatible. Examples of suitable materials include, without limitation, stainless steel, nickel titanium (NiTi) alloys (such as NITINOL) and other shape memory and/or superelastic materials, MP35N, gold, cobalt-chromium alloys, tantalum, platinum or platinum iridium, or other biocompatible metals and/or alloys such as carbon or carbon fiber, cellulose acetate, cellulose nitrate, silicone, cross-linked polyvinyl alcohol (PVA) hydrogel, cross-linked PVA hydrogel foam, polyurethane, polyamide, styrene isobutylene-styrene block copolymer (Kraton), polyethylene terephthalate, polyurethane, polyamide, polyester, polyorthoester, polyanhydride, polyether sulfone, polycarbonate, polypropylene, high molecular weight polyethylene, polytetrafluoroethylene, or other biocompatible polymeric material, or mixture of copolymers thereof, or stainless steel, polymers, and any suitable composite material. A radially self-expanding frame is preferably formed from a shape memory and/or superelastic materials, polymers, or composite material, such as the nickel-titanium alloy NITINOL. The frame can comprise a light-transparent or light-transmitting material such as poly(methylmethacrylate), or halogenated derivates thereof.

The frame may also comprise a bioabsorbable material that can be degraded and absorbed by the body over time to advantageously eliminate a frame structure from the vessel before, during or after the remodeling process. A number of bioabsorbable homopolymers, copolymers, or blends of bioabsorbable polymers are known in the medical arts. These include, but are not necessarily limited to, polyesters including poly-alpha hydroxy and poly-beta hydroxy polyesters, polycaprolactone, polyglycolic acid, polyether-esters, poly(p-dioxanone), polyoxaesters; polyphosphazenes; polyanhydrides; polycarbonates including polytrimethylene carbonate and poly(iminocarbonate); polyesteramides; polyurethanes; polyisocyantes; polyphosphazines; polyethers including polyglycols polyorthoesters; epoxy polymers including polyethylene oxide; polysaccharides including cellulose, chitin, dextran, starch, hydroxyethyl starch, polygluconate, hyaluronic acid; polyamides including polyamino acids, polyester-amides, polyglutamic acid, poly-lysine, gelatin, fibrin, fibrinogen, casein, collagen. The frame may also comprises a nonbioabsorbable homo- or co-polymers such as vinyl polymers including polyfumarate, polyvinylpyrolidone, polyvinyl alcohol, poly-N-(2-hydroxypropyl)-methacrylamide, polyacrylates, and polyalkylene oxalates.

Suitable support frames can also have a variety of configurations, including a plurality of struts and bends forming an array of longitudinally connected hoops, or the frame may comprises braided or helically wound strands. Frames may be formed by cutting from solid tubes, weaving or folding frame members, strands or sheets of material. The support frame can have any suitable size. The exact configuration and size chosen will depend on several factors, including the desired delivery technique, the nature of the body vessel in which the medical device will be implanted, and the size of the vessel. The support frame can be sized so that the second, expanded configuration is slightly larger in diameter that the inner diameter of the vessel in which the medical device will be implanted.

The frame may be sized for implantation within a body vessel using a delivery catheter selected by one skilled in the art for a given application. For example, support frames can be sized and configured for delivery using a delivery catheter selected from one or more delivery catheter sizes from the group consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30 French (F) delivery catheters, or increments of 0.1 F therebetween. In some embodiments, a delivery catheter sized between 1 and 25 F, or preferably between about 1.5 F and 5 F can be used, preferably a 1.8 F (0.60 mm), 2.0 F (0.66 mm), 2.3 F (0.75 mm), 2.6 F (0.85 mm), 2.7 F (0.9 mm), 2.9 F (0.95 mm), or 3.3 (1.10 mm) delivery catheters.

Medical Device Delivery Systems

Medical devices can be delivered into a body lumen using a delivery system which includes a catheter. FIG. 5A depicts a side view of an illustrative preferred embodiment of medical device delivery system 400. The delivery system 400 includes delivery catheter 450 having inner lumen 427 through which inner member 416 is inserted therethrough. A Luer-lock connecter hub 552 may be affixed to the proximal end of the delivery catheter. A Touhy-Borst Y-adaptor or other connector 438 may be connected to the connector hub of the delivery catheter 450 through which inner member 416 is also inserted and extended therethrough. Threaded lock 453 at the proximal end of Y-connector 438 may be rotated to fixedly position inner member 416 longitudinally with respect to the delivery catheter and the Y-connector 438. Side arm 440 of the Y-connector 438 may extend laterally at an acute angle proximally from main arm 439. Alternatively, a commercially available Check-Flo valve may be connected to connector hub 452. This valve is available from Cook Inc., Bloomington, Ind.

FIG. 5B depicts an exploded and enlarged view of delivery catheter 450 and inner member 416 of delivery system 400 of FIG. 5A. Inner member or dilator 416 may be insertable through inner lumen 427 of delivery catheter 450 that extends longitudinally between proximal end 413 and distal end 414. In particular, the inner member 416 is also insertable through, and translatable within, the catheter 450. Inner member 416 includes a distal tip portion 417, proximal portion 418 and intermediate portion 419 disposed between the distal tip portion 417 and the proximal portion 418. A medical device such as a valve or stent graft is positioned around the intermediate portion 419 of the inner member 416 to create a device-containing region 415 adjacent distal end 414. For example, the medical device may be a valve including leaflets formed from lyophilized tissue material that is affixed to a collapsible wire support frame (not shown). The valve leaflets may define a valve orifice through which the intermediate portion of the inner member may be inserted therethrough and engage the tissue material when the valve is positioned around the intermediate portion 419 in a radially compressed configuration. The support frame may be self-expanding and the valve may be maintained in this compressed state by sliding the delivery catheter 450 over the inner member 416 at the device containing region 415 defined within a portion of the lumen 427. The valve is held in the compressed configuration by contact with the inner surface of the catheter 450. The assembled medical device delivery system 400 has inner member 416 extending through inner lumen 427 and beyond the distal end of delivery catheter 450. The compressed medical device is contained in device-containing region 415 of the delivery catheter adjacent distal end 414 of the delivery catheter. The compressed valve may be deployed at the desired vascular site by translating the catheter 450 relative to the inner member 416 by moving one or both of these portions of the system 400. Preferably, the inner member 416 is maintained in a stationary position while withdrawing or pulling back the catheter 450 from the intermediate portion 419 of the inner member 416, exposing the device-containing region 415 to permit radial expansion of the medical device. In other embodiments, the medical device is radially expanded by a balloon within the device-containing region 415. The balloon may be a portion of the inner member 416.

Optionally, the delivery system 400 may be adapted to contact a medical device with fluid within a portion of the delivery system 450. For example, the delivery system 400 may be adapted to hydrate a portion of a medical device containing a dry extracellular matrix material prior to deploying the medical device within the body vessel. A fluid irrigation source may be connected to side arm 440 via well-known inner interconnecting tubing and valve 454. The side arm of the Y-adaptor or connector permits the delivery of a fluid to hydrate lyophilized material (e.g, ECM or other tissue) of the medical device contained in the device-containing region 415 of the delivery catheter adjacent the distal end thereof. The medical device that is contained in the delivery system of the present invention is typically delivered percutaneously to a vascular deployment site over a well-known wire guide that is inserted into the vascular system and to the deployment site. Inner lumen 24 extends longitudinally through inner member 16 as well as the delivery system, which is introduced over the wire guide through the inner lumen. The system 400 preferably includes a fluid delivery lumen extending from the proximal end of the system 424 to the device-containing region 415. For example, proximal portion 418 of inner member 416 may have a cylindrical outer surface 434 with a recessed or flat portion 435 extending longitudinally therealong and communicating with intermediate portion 419. This recessed or flat portion 435 of the inner member 416 in combination with delivery catheter 450 may form a portion of a fluid delivery lumen 441 through which a fluid may be transported to hydrate a portion of the medical device contained in the device containing region 415 of the intermediate portion 419 of the inner member 416.

A reduced diameter intermediate portion 419 of the inner member 416 may be inserted through and engage the medical device for centering the medical device in the vessel in which the device is being deployed. To maintain the longitudinal position of the medical device in the vessel during deployment, proximal portion 418 of the inner member may include a blunt distal end 420 to engage the proximal end of the medical device positioned in the intermediate portion 419. This blunt distal end 420 may also be effective in holding, for example, a medical device configured as a stent or stent graft that can be contained in the device-containing region 415 and intermediate portion 419. The blunt distal end preferably closely approximates the size of inner lumen 427 of delivery catheter 450.

Distal tip portion 417 of inner member 416 may include a tapered proximal end 431, tapered distal end 432 and an intermediate segment 433 disposed between the tapered distal and proximal ends. The tapered distal end facilitates atraumatic placement of the delivery system to the deployment site. Tapered proximal end 431 advantageously provides atraumatic withdrawal of the inner member through the valve slit or opening after the stent valve has been deployed at the desired vessel site. Intermediate segment 433 approximates the size and shape of inner lumen 427 of delivery catheter 450 so as to provide an atraumatic transition between the assembled inner member and delivery catheter. Optionally, the distal tip portion 417 may include a means for locating or orienting the system 400, such as a band of radiopaque marker material.

FIG. 5C depicts an enlarged partial and longitudinally sectioned view of the delivery system 400 of FIG. 5A with inner member 416 inserted within the catheter 450. Delivery catheter 450 comprises a cylindrical tube of, for example, a radiopaque fluorinated ethylene propylene material with a device-containing region 415 of a cylindrical tube of a clear or transparent non-radiopaque fluorinated ethylene propylene material thermally bonded to radiopaque tube at the distal end thereof. The two tubes may be thermally bonded at a tapered bonding area 423. The strength of the thermal bond may be improved by tapering the one end of one tube and correspondingly beveling the matching end of the other tube. The transparent tube may be configured to permit visual inspection of the medical device such as a venous valve contained within region 415 of the delivery catheter 450 and around the recessed portion of inner member 416. The hydration of a medical device within the device-containing region 415 may be verified through the transparent tube portion of the catheter 450 prior to implantation. In addition, a visual check can also be made to verify the absence of air emboli in the catheter. Coaxial portions of the inner member 416 may be reduced in its outer diameter by about one French size so as to create an approximately 0.017 to 0.018 inch difference from the outer catheter 450 portion.

Methods of Treatment

Still other embodiments provide methods of treating a subject, which can be animal or human, comprising the step of implanting one or more medical devices comprising a transmitting surface and an adhesive surface, as described herein. The medical device is preferably a valve comprising one or more valve leaflets, or a stent graft, and may optionally comprise a support frame. In some embodiments, methods of treating may also include the step of delivering a medical device to a point of treatment in a body vessel, or deploying a medical device at the point of treatment. The medical device can be implanted in any suitable body vessel, including a vein or artery. The configuration of the implantable medical device can be selected based on the desired site of implantation. For example, for implantation in the superficial artery, popliteal artery or tibial artery, frame designs with increased resistance to crush may be desired. For implantation in the renal or iliac arteries, frame designs with suitable levels of radial force and flexibility may be desired.

Methods for delivering a medical device to any suitable body vessel are also provided, such as a vein, artery, biliary duct, ureteral vessel, body passage or portion of the alimentary canal. While many preferred embodiments discussed herein discuss implantation of a medical device in a vein, other embodiments provide for implantation within other body vessels. In another matter of terminology there are many types of body canals, blood vessels, ducts, tubes and other body passages, and the term “vessel” is meant to include all such passages.

In some embodiments, medical devices can be intraluminally delivered inside the body by a delivery system including a catheter configured to support the medical device in a radially compressed configuration as it is transported to the desired site, for example within a body vessel. Upon reaching the site, the medical device can be deployed. The medical device may be deployed by forcing the stent to expand radially outward, for example by inflation of a balloon positioned in the distal portion of the catheter. The radially expanded medical device may be fixed in an expanded configuration in contact with the lumen wall, and securably placed within the body vessel, for example by activating a light-activated adhesive on an adhesive surface of the medical device. When the delivery process comprises inflation of a balloon, the balloon can be subsequently deflated and the delivery catheter removed.

In another delivery technique, the medical device is formed of an elastic material that will self-expand after being compacted. During introduction into the body, the medical device is restrained in the compressed configuration. When the medical device has been delivered to the desired site for implantation, the restraint is removed, allowing the medical device to self-expand by its own internal elastic restoring force. Once the medical device is located at the constricted portion of the lumen, the sheath is moved longitudinally away from the medical device, permitting the medical device to expand until it contacts the body vessel wall. Preferably, the delivery catheter is subsequently removed from the body vessel without moving the implanted medical device.

In a first aspect, methods of treating venous valve related conditions are provided. A “venous valve related condition” is any condition presenting symptoms that can be diagnostically associated with improper function of one or more venous valves. In mammalian veins, natural valves are positioned along the length of the vessel in the form of leaflets disposed annularly along the inside wall of the vein which open to permit blood flow toward the heart and close to prevent back flow. These natural venous valves act as open to permit the flow of fluid in the desired direction, and close upon a change in pressure, such as a transition from systole to diastole. When blood flows through the vein, the pressure forces the valve leaflets apart as they flex in the direction of blood flow and move towards the inside wall of the vessel, creating an opening therebetween for blood flow. The leaflets, however, do not normally bend in the opposite direction and therefore return to a closed position to restrict or prevent blood flow in the opposite, i.e. retrograde, direction after the pressure is relieved. The leaflets, when functioning properly, extend radially inwardly toward one another such that the tips contact each other to block backflow of blood. Two examples of venous valve related conditions are chronic venous insufficiency and varicose veins.

In the condition of venous valve insufficiency, the valve leaflets do not function properly. For example, the vein can be too large in relation to the leaflets so that the leaflets cannot come into adequate contact to prevent backflow (primary venous valve insufficiency), or as a result of clotting within the vein that thickens the leaflets (secondary venous valve insufficiency). Incompetent venous valves can result in symptoms such as swelling and varicose veins, causing great discomfort and pain to the patient. If left untreated, venous valve insufficiency can result in excessive retrograde venous blood flow through incompetent venous valves, which can cause venous stasis ulcers of the skin and subcutaneous tissue. Venous valve insufficiency can occur, for example, in the superficial venous system, such as the saphenous veins in the leg, or in the deep venous system, such as the femoral and popliteal veins extending along the back of the knee to the groin.

The varicose vein condition consists of dilatation and tortuousity of the superficial veins of the lower limb and resulting cosmetic impairment, pain and ulceration. Primary varicose veins are the result of primary incompetence of the venous valves of the superficial venous system. Secondary varicose veins occur as the result of deep venous hypertension which has damaged the valves of the perforating veins, as well as the deep venous valves. The initial defect in primary varicose veins often involves localized incompetence of a venous valve thus allowing reflux of blood from the deep venous system to the superficial venous system. This incompetence is traditionally thought to arise at the saphenofemoral junction but may also start at the perforators. Thus, gross saphenofemoral valvular dysfunction may be present in even mild varicose veins with competent distal veins. Even in the presence of incompetent perforation, occlusion of the saphenofemoral junction usually normalizes venous pressure.

The initial defect in secondary varicose veins is often incompetence of a venous valve secondary to hypertension in the deep venous system. Since this increased pressure is manifested in the deep and perforating veins, correction of one site of incompetence could clearly be insufficient as other sites of incompetence will be prone to develop. However, repair of the deep vein valves would correct the deep venous hypertension and could potentially correct the secondary valve failure. Apart from the initial defect, the pathophysiology is similar to that of varicose veins.

In a second aspect, methods of treating peripheral vascular disease, including critical limb ischemia, are provided. Atherosclerosis underlies most peripheral vascular disease. Narrowed vessels that cannot supply sufficient blood flow to exercising leg muscles may cause claudication, which is brought on by exercise and relieved by rest. As vessel narrowing increases, critical limb ischemia (CLI) can develop when the blood flow does not meet the metabolic demands of tissue at rest. While critical limb ischemia may be due to an acute condition such as an embolus or thrombosis, most cases are the progressive result of a chronic condition, most commonly atherosclerosis. The development of chronic critical limb ischemia usually requires multiple sites of arterial obstruction that severely reduce blood flow to the tissues. Critical tissue ischemia can be manifested clinically as rest pain, nonhealing wounds (because of the increased metabolic requirements of wound healing) or tissue necrosis (gangrene).

The medical devices can be placed in any medically appropriate location for a given application. For example, in some embodiments, the medical device can serve as part of a venous valve prosthetic and be implanted in the femoral vein, including at the proximal (groin), mid (mid section) or distal (adjacent to the knee) portions of the vein. The medical device can be preferably implanted within the tibial arteries for treatment of CLI. For instance, the medical device can be configured as a vascular stent having a self-expanding support frame formed from a superelastic self-expanding nickel-titanium alloy attached to a covering material. The use of a self-expanding frame can be preferably used when the body vessel to be stented extends into the distal popliteal segment. The selection of the type of implantable frame can also be informed by the possibility of external compression of an implant site within a body vessel during flexion of the leg.

### EXAMPLES

### Example 1

### Small Intestine Submucosa Transmitting Surface

The transmissivity of the porcine small intestine submucosa (SIS) samples were measured as a function of the distance of the ultraviolet light source from the SIS sample. SIS is one example of a preferred extracellular matrix material suitable for forming a transmitting surface in an endolumenal valve.

A UV-light source provided suitable adhesive-activating light at 405 nm with a typical output of about 500 mW/cm² (Loctite 7700, Rocky Hill, Conn.) (“light source”). The irradiance of the UV-light source was measured using a Loctite Zeta 7011-A Dosimeter-Radiometer (Loctite, Rocky Hill, Conn.) (“detector”). Irradiance is a measure of the amount of light energy incident on a unit area of surface per unit time. The irradiance of the UV-light source was measured through air as a function of distance between the light source and the detector, and the results are shown in Table 1 below.

TABLE 1

| Distance | Dose | Irradiance |
|---|---|---|
| (mm) | (J/cm²) | (mW/cm²) |
| 0 | 2.28 | 234 |
| 5 | 0.47 | 066 |
| 10 | 1.91 | 89 |
| 15 | 0.34 | 36 |
| 20 | 0.34 | 36 |
| 25 | 0.28 | 27 |
| 30 | 0.18 | 18 |

Two 1 cm² samples of dry SIS (Cook Incorporated, Bloomington, Ind.) were weighed and hydrated by placing the material in saline solution for 5 minutes at room temperature. The thickness and weight of each dry sample was recorded prior to hydration, and each sample was weighed in the hydrated state. Results from these measurements are provided in Table 2 below.

TABLE 2

| Sample | Thickness Dry | Weight Dry | Weight Hydrated |
|---|---|---|---|
| 1 | 0.0015-inch | 6.3 mg | 45 mg |
| 2 | 0.0020-inch | 2.4 mg | 36 mg |

The hydrated samples were separately placed over the detector (Loctite Zeta 7011-A Dosimeter-Radiometer) and the transmissivity of each hydrated sample was recorded as a function of the distance between the sample and the UV-light source. Each SIS sample was wet in saline prior to measurements and then placed on the dosimeter sensor window and both were positioned at a distance from 0 to 30 mm from the UV source in increments of 5 mm. Results from these measurements of Sample 1 are recorded in Table 3 below, and in Table 4 for Sample 2.

TABLE 3
Sample 1 0.0015-inch thick SIS

| Distance | Dose | Irradiance |
|---|---|---|
| (mm) | (J/cm²) | (W/cm²) |
| 0 | 0.31 | 0.047 |
| 5 | 0.81 | 0.018 |
| 10 | 0.32 | 0.03 |
| 15 | 0.13 | 0.012 |
| 20 | 0.09 | 0.008 |
| 25 | 0.06 | 0.005 |
| 30 | 0.04 | 0.004 |

TABLE 4
Sample 2 0.0020-inch thick SIS

| Distance | Dose | Irradiance |
|---|---|---|
| (mm) | (J/cm²) | (W/cm²) |
| 0 | 0.35 | 0.035 |
| 5 | 0.38 | 0.036 |
| 10 | 0.34 | 0.032 |
| 15 | 0.2 | 0.016 |
| 20 | 0.12 | 0.01 |
| 25 | 0.1 | 0.009 |
| 30 | 0.06 | 0.006 |

The irradiance of both SIS samples decreased with increasing distance from the UV source. At 0 mm from the light source Sample 1 had the higher irradiance when compared to the SIS from the ICLD valve (0.047 W/cm² and 0.035 W/cm², respectively, corresponding to a percent difference of 25.5%). At 30 mm from the light source, Sample 2 had the lower irradiance when compared to the SIS from the ICLD valve (0.004 W/cm² and 0.006 W/cm², respectively, a difference of 33.3%). For comparison, the irradiance emitted without SIS is 0.234 W/cm² at 0 mm and 0.018 W/cm² at 30 mm from the light source. A maximum irradiance of 0.0476 W/cm² and a minimum of 0.004 W/cm² were measured through the SIS samples. The control values of irradiance measured though air ranged from a maximum of 0.234 W/cm² to a minimum of 0.018 W/cm².

### Example 2

### Activation of an Adhesive through a Small Intestine Submucosa Transmitting Surface

An adhesive was activated by ultraviolet light through samples of porcine small intestine submucosa (SIS) at two different thicknesses. A UV-light activated cyanoacrylate ultraviolet adhesive (4304 Flashcure® Light Cure Adhesive, Loctite, Rocky Hill, Conn.), was activated through a hydrated SIS material described as Sample 1 in Example 1 above. A small drop of the adhesive was placed on a glass slide. The 405 nm UV-light (Loctite 7700 curing light used in Example 1) was shone through the hydrated 1 cm² SIS material of Sample 1 and directed at the adhesive. The UV light source and SIS were placed approximately 5 mm from the adhesive. After approximately 10 seconds, the adhesive was fully set (i.e., activated).

### Prophetic Example 1

### Preparation of a UV-Activated Adhesive Comprising a Photoreactive Gelatin

4-Benzoylbenzoic acid (1.7 g, 7.7 mmol, Aldrich Chemical Company, Inc., Wis.) can be dissolved into 10 mL of an aqueous sodium hydroxide solution (1.0 N) and then neutralized to about pH 8 with 6.0 N and 1.0 N hydrochloric acid. To the aqueous solution, 60 mL of a phosphate-buffered saline solution (PBS; pH 7.4) of 1-ethyl-3-(3-dimethylaminopropyl)carboniimide hydrochloride (2.9 g, 15.3 mmol) can be added, and the mixture can be stirred for 30 minutes at 0° C. and for an additional 10 minutes at room temperature. After the addition of PBS (20 mL) and gelatin (2.0 g from bovine bone, M_{w}=95000), the mixed solution is stirred overnight at room temperature. The resulting reaction mixture is dialyzed using a seamless cellulose tube (Dialysis Membrane, size 36) in de-ionized water for 3 days and then lyophilized using a freeze dryer under reduced pressure to yield a benzophenone-derived gelatin as a white powder.

Xanthene dye-derivatized gelatins can be synthesized by a similar procedure. PBS (10 mL) of fluorescein sodium salt (2.9 g, 7.7 mmol) and PBS (60 mL) of 1-ethyl-3-(3-dimethylaminopropyl)carbodiimide hydrochloride (2.9 g, 15.3 mmol) are mixed together while stirring at 0° C. for 20 minutes and at room temperature for 10 minutes, and then to the mixed solution PBS (20 mL) of gelatin (2.0 g) is added. After stirring at room temperature overnight, the fluorescein-derived gelatin can be isolated from dialysis and subsequent lyopholization as a yellow powder.

Photocurable, UV-light-activated adhesives can be prepared by dissolving the photoreactive gelatin in a saline solution at different concentrations with or without poly(ethylene glycol) diacrylate PEGDA. The adhesives containing a dye-derivatized gelatin can be mixed with 0.3 wt % of ascorbic acid as a reducing agent.

The invention includes other embodiments within the scope of the claims, and variations of all embodiments, and is limited only by the claims made by the Applicants.

We claim:

1. An endolumenal medical device delivery system comprising:
a. a light source adapted to emit light at a first wavelength and
b. an endoluminal medical device having an interior surface defining a lumen and an exterior surface, the endolumenal medical device including:
i. a light-transmitting area positioned between the interior surface and the exterior surface, the light-transmitting area having an adhesive-activating transmittance at the first light wavelength; and
ii. a light-activated adhesive positioned and configured to permit light passing through the transmitting area to contact the light-activated adhesive, the light-activated adhesive being activated by the light at the first wavelength.

2. The endolumenal medical device delivery system of claim 1, wherein the light-activated adhesive is in contact with at least a portion of the exterior surface.

3. The endolumenal medical device delivery system of claim 1, wherein the light source includes a catheter containing a light-emitting portion.

4. The endolumenal medical device delivery system of claim 1, wherein the first wavelength is ultraviolet, visible or infrared light.

5. The endolumenal medical device delivery system of claim 1, wherein the first wavelength is between about 200 nm and about 450 nm.

6. The endolumenal medical device delivery system of claim 1, wherein the light-activated adhesive comprises a material selected from the group consisting of: N-vinyl pyrollidone (NVP), 2-acrylamido-2-methyl-1-propane sulfonic acid (AMPS), vinylsuccinimide (VS), 2-isocyanatoethyl methacrylate (IEM) or glycidyl acrylate (GA), poly(ethylene glycol) (PEG), poly(propylene glycol) (PPG), benzophenone, xanthene dye, poly(ethylene glycol)diacrylate (PEGDA), 2,2-bis(4-ethoxyacrylatephenyl)propane (HEPAC), 2,2-bis(4-ethoxyacrylatephenyl)6-Fpropane (HEPFAAC), 2,2-bis(4-ethoxyacrylate-3,5-dibromophenyl)propane (TBHEPAAC), 5-tert-butyl-1,2-bis(4-ethoxy-acrylate benzoyl)benzene (tBuHEPBAC), gelatin, cinnamate, coumarin and thymine, or a copolymer of two or more of the materials.

7. The endolumenal medical device delivery system of claim 1, wherein the endolumenal medical device is configured as a stent graft comprising graft material forming the light-transmitting area.

8. The endolumenal medical device delivery system of claim 1, wherein the endolumenal medical device is configured as a radially-expandable endolumenal valve.

9. The endolumenal medical device delivery system of claim 8, wherein the endolumenal medical device is adapted for endovascular implantation in a vein or a heart.

10. The endolumenal medical device delivery system of claim 1, wherein the endolumenal medical device is configured as a valve comprising a valve leaflet forming the transmitting surface.

11. The endolumenal medical device delivery system of claim 1, wherein the endolumenal medical device includes an extracellular matrix material forming at least a portion of the light-transmitting area.

12. The endolumenal medical device delivery system of claim 1, wherein the endolumenal medical device is a valve including at least one leaflet comprising small intestine submucosa forming at least a portion of the light-transmitting area.

13. The endolumenal medical device delivery system of claim 1, wherein:
a. the light source is a catheter comprising an expandable balloon, a light source and a light-transmitting portion, the catheter adapted to emit light at the first wavelength within a body lumen; and
b. the endolumenal medical device is a radially expandable valve comprising a support frame and a valve leaflet attached to the support frame, the valve leaflet defining a portion of the exterior surface and a portion of the interior surface and including a portion of the light-transmitting area, and a portion of the interior surface of the radially expandable valve circumferentially enclosing a portion of the catheter.

14. The endolumenal medical device delivery system of claim 13, wherein the light-activated adhesive is also in contact with a portion of the support frame.

15. The endolumenal medical device delivery system of claim 13, wherein the light-activated adhesive is in contact with the valve leaflet.

16. The endolumenal medical device delivery system of claim 13, wherein the first wavelength is ultraviolet, visible or infrared light.

17. A method of manufacturing a radially expandable medical device comprising:
a. attaching at least one light-transmitting material to a radially expandable support frame, the light-transmitting material comprising an extracellular matrix material forming a transmitting area having an adhesive-activating transmittance at a first light wavelength; the valve being moveable from a radially compressed delivery configuration to a radially expanded configuration;
b. contacting the transmitting area of the extracellular matrix material of the valve leaflet with a hydrating fluid in a manner effective to absorb the hydrating fluid into the extracellular matrix material to form a hydrated valve leaflet; and
c. applying a light-activated adhesive to at least a portion of the support frame or the hydrated valve leaflet to form an adhesive surface comprising the light-activated adhesive positioned and configured to permit light passing through the light-transmitting area contacts the adhesive surface.

18. A method of treatment comprising the steps of:
a. positioning an endolumenal medical device having an interior surface defining a lumen and an exterior surface in contact with a body vessel, the medical device comprising a light-transmitting area and a light-activated adhesive, the light-transmitting area having an adhesive-activating transmittance at a first light wavelength; the light-activated adhesive positioned and configured to permit light passing through the light-transmitting area to contact the light-activated adhesive, the light-activated adhesive being activated by light of the first wavelength; and
b. illuminating the light-activated adhesive within the body vessel with light of the first light wavelength in a manner effective to activate the light-activated adhesive within the body vessel.

19. The method of claim 18, wherein the light-activated adhesive contacts the wall of the body vessel after positioning the endolumenal medical device within the body vessel and illuminating the light-activated adhesive secures a portion of the light-transmitting area to the wall of the body vessel.

20. The method of claim 18, wherein the endolumenal medical device is a first endolumenal medical device, the light-activated adhesive is positioned on the interior surface of the endolumenal medical device and the method further comprises the steps of:
c. inserting a second endolumenal medical device into the body vessel and contacting the second endolumenal medical device with the light-activated adhesive on the interior surface of the first endolumenal medical device; the step of illuminating the light-activated adhesive being effective to join the first endolumenal medical device to the first endolumenal medical device; and
d. removing the first endoluminal device and the second endolumenal medical device from the body vessel.
